(19) United States
(12) Patent Application Publication
(10) Pub. No.: US 2006/0206138 A1
(43) Pub. Date: Sep. 14, 2006

(54) Intravascular filter assembly

(76) Inventor: Tracee E.J. Eidenschink, Wayzata, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/075,923

(22) Filed: Mar. 9, 2005

(51) Int. Cl. A61M 29/00

(52) U.S. Cl. 606/200

(57) ABSTRACT

An intravascular filter assembly for use in a body vessel lumen having a wall, which may include a first filter having a mouth facing in a first direction at a first location for receiving emboli and an end in a second direction opposite the first direction, and a second filter having a mouth facing in the first direction at a first location for receiving emboli and an end in the second direction, wherein the second filter is removably attached to the first filter.

### FIELD OF THE INVENTION

The present invention pertains to embolic protection. More particularly, the present invention pertains to embolic protection filters and methods of making the same.

### BACKGROUND OF THE INVENTION

There are a number of situations in the practice of medicine when it becomes desirable for a physician to place a filter in the vascular system of a patient. One of the most common applications for vascular filters is the treatment of Deep Venous Thrombosis (DVT). Deep Venous Thrombosis patients experience clotting of blood in the large veins of the lower portions of the body. These patients are constantly at risk of a clot breaking free and traveling via the inferior vena cava to the heart and lungs. This process is known as pulmonary embolization. Pulmonary embolization can frequently be fatal, for example when a large blood clot interferes with the life-sustaining pumping action of the heart. If a blood clot passes through the heart it will be pumped into the lungs and may cause a blockage in the pulmonary arteries. A blockage of this type in the lungs will interfere with the oxygenation of the blood causing shock or death.

Pulmonary embolization may be successfully prevented by the appropriate placement of a thrombus filter in the vascular system of a patient's body. Placement of the filter may be accomplished by performing a laparotomy with the patient under general anesthesia. However, intravenous insertion is often the preferred method of placing a thrombus filter in a patient's vascular system.

Intravenous insertion of a thrombus filter is less invasive and it requires only a local anesthetic. In this procedure, the thrombus filter is collapsed within a delivery catheter. The delivery catheter is introduced into the patients vascular system at a point which is convenient to the physician. The delivery catheter is then fed further into the vascular system until it reaches a desirable location for filter placement. The thrombus filter is then released into the blood vessel from the delivery catheter.

In the treatment of Deep Venous Thrombosis, a thrombus filter is placed in the inferior vena cava of a patient. The inferior vena cava is a large vessel which returns blood to the heart from the lower part of the body. The inferior vena cava may be accessed through the patient's femoral or jugular vein.

Thrombus filters may be placed in other locations when treating conditions other than deep venous thrombosis. For example, if blood clots are expected to approach the heart and lungs from the upper portion of the body, a thrombus filter may be positioned in the superior vena cava. The superior vena cava is a large vessel which returns blood to the heart from the upper part of the body. The superior vena cava may also be accessed through the jugular vein or femoral vein.

Once placed inside a blood vessel, a thrombus filter acts to catch and hold blood clots. The flow of blood around the captured clots allows the body's lysing process to dissolve the clots.

It is recognized in the art that it is undesirable for a thrombus filter to change position once it has been place in the desired position by a physician. If a filter becomes loose in the lumen of a blood vessel, it may migrate to a position where it may be ineffective at capturing thrombi. Alternately, and more seriously, a loose thrombus filter may migrate to a dangerous or life threatening position. Prior art filters have addressed this concern by including anchor members which penetrate the vessel walls.

The walls of the blood vessels are lined with a thin inner membrane which may be referred to as the intima or the endothelium. When this inner membrane is disrupted by a foreign object such as a thrombus filter the body responds in a process referred to as neointimal hyperplasia. As a result, the disrupted area of inner membrane is overgrown with a number of new cells. The anchor portions of the thrombus filter are encapsulated with new cell growth, sometimes referred to as endothelial growth. Removing the filter once the anchor portions have been encapsulated may result in trauma to the vessel wall. There is thus an ongoing need to provide alternative designs and methods for making and using medical devices with desirable characteristics and features.

### SUMMARY OF THE INVENTION

One embodiment of the invention pertains to an intracorporal filter assembly including two or more filters removably attached to each other. One filter may have anchoring members for anchoring the filter to a vessel wall. A second filter may be removably attached to the first filter and may have ends configured to rest against the wall of the vessel. For example, the first filter may have a tapered elongate member extending from a central portion and the second filter may have a lumen for receiving the end of this elongate member. The ends may be wide or curved inward to reduce the chance of encapsulation in the vessel wall. The second filter may also have hooks or another mechanism by which the second filter may be grasped for collapse and removal. The second filter may be spaced apart from the first. The first filter and the second filter may include elongate legs for filtering. The legs of the second filter may be radially offset from the legs of the first filter.

Another embodiment of the invention pertains to an intracorporal filter assembly including two or more filters removably attached to each other by an elongate member. The first filter may have anchoring members for attaching the assembly to a vessel wall. The second and subsequent filters may be configured so they do not touch the vessel wall when installed. The filters may be spaced apart or more closely stacked.

Another embodiment of the invention pertains to an intracorporal filter assembly including two or more filters stacked together. A first filter may include anchoring members for fixing the filter to a vessel wall. A second filter and a third filter may rest in the cavity of the first filter and held in place by the blood flow.

Another embodiment pertains to a method of using an intracorporal filter. For example, a filter assembly having a three filters may be installed in a body vessel lumen such that the first filter is anchored to the wall of the vessel lumen. The filters, predominately the third filter, trap emboli. A retrieval catheter is inserted into the vessel lumen and the third filter is collapsed and retrieved, leaving the first and second filters. The second filter may be retrieved in a similar manner, if desired.

Another embodiment pertains to a method of

The above summary of some embodiments is not intended to describe each disclosed embodiment or every implementation of the present invention. The figures and detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings in which:

FIG. 1 is a perspective view of an intracorporal filter assembly 100;

FIG. 2 is a diagrammatic view of an intracorporal filter assembly 200;

FIG. 3 is a diagrammatic view of an intracorporal filter assembly 300;

FIG. 4 is a diagrammatic view of an intracorporal filter assembly 400;

FIG. 5 is a diagrammatic view of a filter 402 of the assembly 400;

FIG. 6 is a diagrammatic view of an intracorporal filter assembly 500;

FIG. 7 is a diagrammatic view of an intracorporal filter assembly 600 loaded in a delivery device;

FIG. 8 is a diagrammatic view of assembly 600 being installed in a body vessel lumen;

FIG. 9 is a diagrammatic view of a filter 604 being withdrawn from assembly 600; and

FIG. 10 is a diagrammatic view of a filter 604 loaded in a delivery device.

### DETAILED DESCRIPTION OF ILLUSTRATED EMBODIMENTS

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

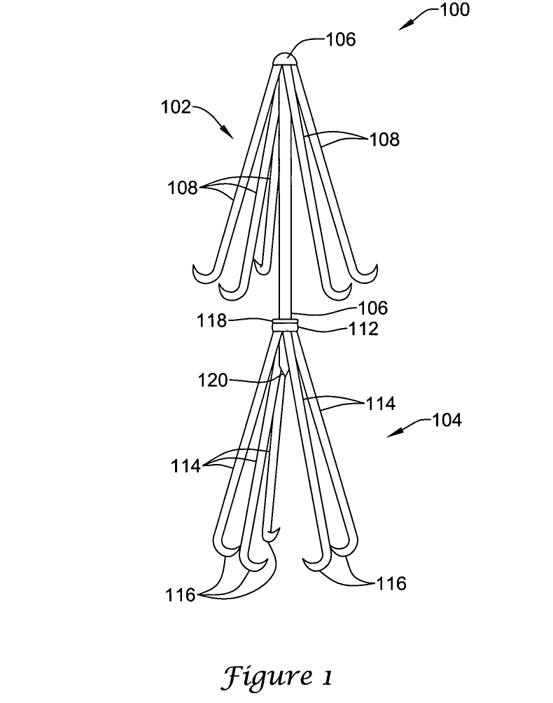

FIG. 1 is a perspective view of an intracorporal filter assembly 100. Assembly 100, like other assemblies of the illustrative embodiments, is depicted as an intravascular filter though the invention is suitable for use in intracorporal filters besides intravascular filters. Assembly 100 includes a first filter 102 and a second filter 104. Filters 102 and 104 are removably connected by an elongate member 106. Filters 102 and 104 are depicted as Greenfield style filters. The invention is suitable with a wide range of filter configurations and should not be construed as limited to this particular filter style. Filters 102 and 104 are depicted with five legs. Other numbers of legs are suitable and are contemplated. For example, filter 102 may include three, six or eight legs, and may have a different number of legs from filter 104. Filter 102 includes an apical head 106, legs 108 extending radially out from apical head 106 and in a first longitudinal direction, and anchoring members 110. Anchoring members 110 are depicted as hooks, but may be any suitable anchoring members as are known by those of skill in the art. Anchoring members 110 are configured to retain filter 102 to the wall of a body vessel lumen. Filter 104 includes apical ring 112, legs 114 extending radially out from apical ring 112 in a first direction and having ends 116 at their termini. Ends 116 are configured to rest against the wall of a body vessel lumen while not triggering encapsulation. They are consequently atraumatic, and in the depicted configuration curve inward. Ends 116 optionally include hooks to aid with the withdrawal of filter 104. Ends 116 may be coated with a therapeutic agent to reduce neointimal hyperplasia.

Elongate member 106 is attached to filter 102 at the apical head 106 and passes through apical ring 106 of filter 104. Elongate member 106 may include a stop 118 and may also include a taper 120. Stop 118 may be used to position filter 104. Other structures which may be suitable include, for example, a stepwise reduction in the diameter of the elongate member or a gradual taper. Alternatively, filter 104 may be fixed to elongate member 106 with a weak adhesive.

Filters 102 and 104 are depicted spaced apart such that filter 104 is in the first direction from the mouth of filter 102. Other configurations are contemplated and the filters could be spaced further apart or closer together. For example, elongate member 106 could be should and filter 104 could be positioned in the mouth of filter 102. The legs of filter 102 are depicted at a first radial angle and the legs of filter 104 are depicted at a second radial angle different from the first. This may be desirable. Alternatively, both sets of legs could be at the same radial angle.

Assembly 100 may be made from a metal such as stainless steel or a nickel titanium alloy. Other metals which may be suitable to use in the filter in whole or part include gold, tantalum, tungsten, titanium, Beta III Titanium, Nitinol, a cobalt cromium alloy, Elgiloy, L605, MP35N, Ta-10W, 17-4PH, or Aeromet 100. Certain polymers such as thermosets or thermopolymers may be suitable as well. The filter legs may be formed from a wire, rod, tubing or other structure that can be processed to form the structures shown.

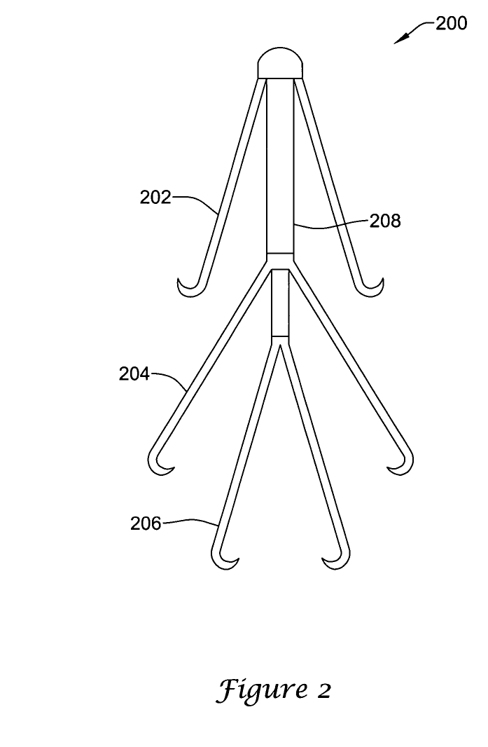

In FIG. 1, two filters are shown. In some contemplated embodiments, three or more filters may be included in the assembly, each filter being releasably connected to its neighbors, as is shown in FIG. 2, which is a diagrammatic side view of an intracorporal filter assembly 200. Each filter in FIG. 2 is depicted with only two legs but may have any number of legs. For example, each filter may have 4 legs. Assembly 200 includes a first filter 202, a second filter 204, a third filter 206, and an elongate member 208. Filter 202 includes apical head 212, legs 214, and anchoring members 216 for anchoring the filter to the wall of a body vessel lumen. Elongate member 208 is attached to apical head 212. Elongate member 208 has two stepwise reductions in diameter, on which filters 204 and 206 are disposed Filters 204 and 206 are releasably attached to the elongate member. For example, they may be abutted against the stepwise reductions or they may be affixed with a weak adhesive. At the first stepwise reduction in diameter is the apical ring 218 of filter 204. Filter 204 also includes legs 220 and atraumatic ends 222. At the second stepwise reduction of the elongate member is the apical ring 224 of filter 206. Filter 206 also include legs 226 and atraumatic ends 228. Filters 202, 204, and 206 are depicted as having the same configuration. However, a variety of filter types may be used as desired. Filters 204 and 206 are spaced from filter 202 while filter 206 nests in filter 204. The filters may be spaced differently, as desired. Moreover, more or fewer filters may be used as desired and the elongate member may be configured accordingly.

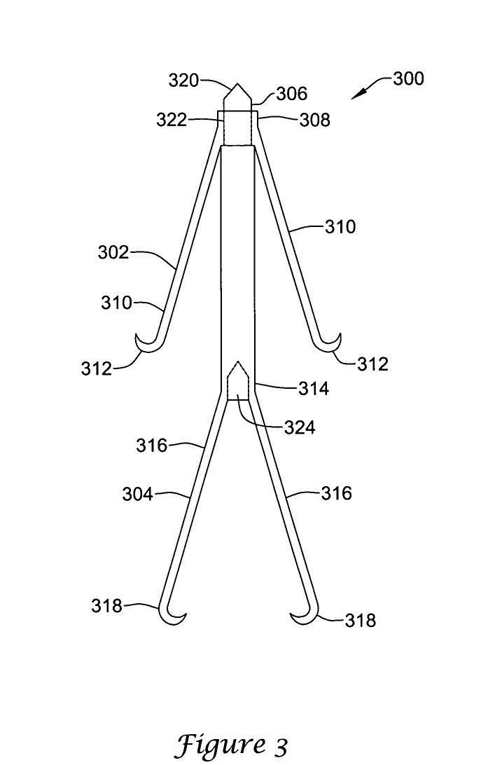

FIG. 3 is a diagrammatic view of an intracorporal filter assembly 300, which includes first filter 302, second filter 304, and elongate member 306. Filter 302 includes apical ring 308, legs 310 and anchoring members 312. Filter 304 includes apical ring 314, leg 316, and atraumatic members 318. Elongate member 306 is attached to filter 302, and may include a taper 320, a stepwise increase in diameter 322, and a cavity 324. The cavity is shaped to receive the tip of a similar elongate member. Elongate member 306 is received into apical ring 308 and is prevented from further penetration by the stepwise increase in diameter. The cavity permits successive filters 304 and elongate members 306 to be added to the assembly.

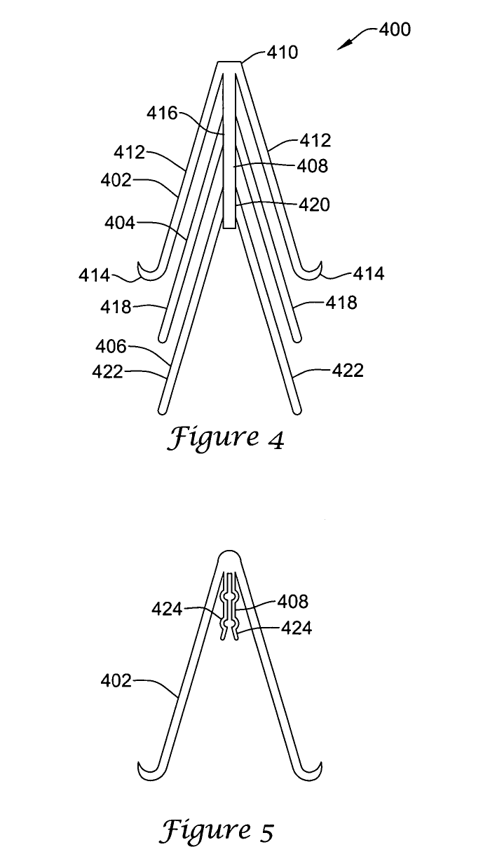

FIG. 4 is a diagrammatic view of an intracorporal filter assembly 400, which includes first filter 402, second filter 404, third filter 406 and elongate member 408. Filter 402 includes apical head 410, legs 412, and anchoring members 414. Filter 404 includes apical head 416 and legs 418 and filter 406 includes apical head 420 and legs 422. Filter 402 is a first diameter of a size so that anchoring members 414 engage the wall of a body vessel lumen. Filters 404 and 406 are a second diameter of a size so that the filters do not touch the walls of the body vessel lumen. Alternatively, filters 404 and 406 are of a size that they slightly graze the walls of the body vessel lumen. Elongate member 408 is attached to filter 402 and receives filters 404 and 406. As can be seen in FIG. 5, which is a side diagrammatic view of filter 402 and elongate member 408, the elongate member includes two flexible fingers 424 which form cavities to receive the apical heads of filters 404 and 406, which may be pushed into the elongate member to releasable attach them to filter 402.

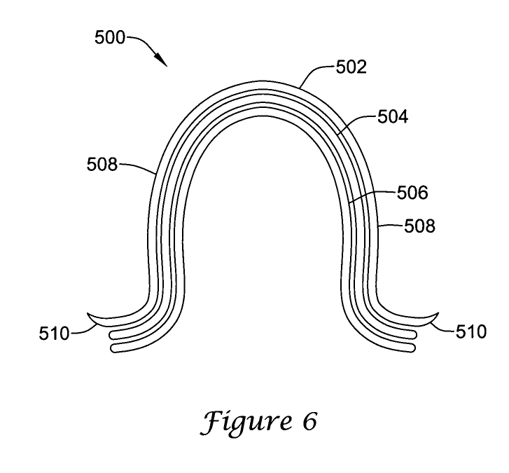

FIG. 6 is a diagrammatic view of an intracorporal filter assembly 500, which includes filters 502, 504, and 506. Filter 502 includes legs 508 and anchoring members 510. Filters 504 and 506 nest closely inside filter 502 and may be held in place by a spring force of filter 504 and 506, by a weak adhesive, or by the direction of the blood flow. Each of filters 502, 504, and 506 may include only three legs or may include another suitable number of legs, and each set of legs may be at a radial angle different from the others.

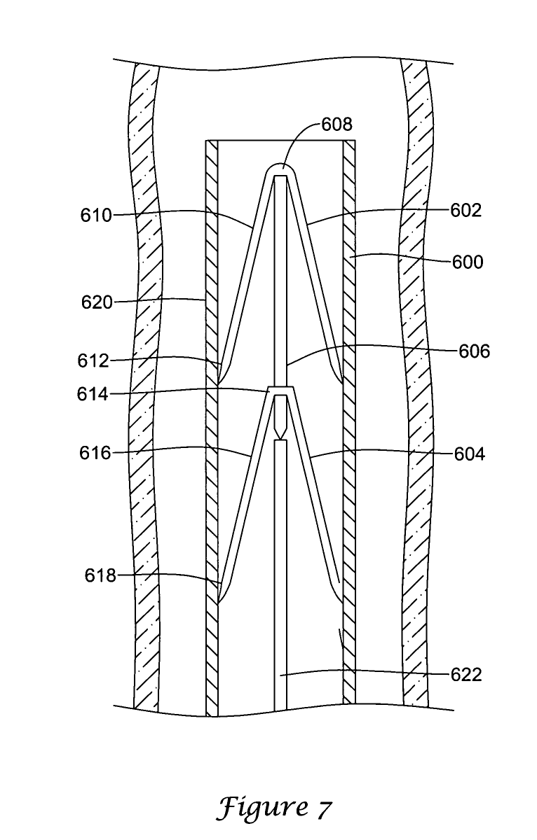

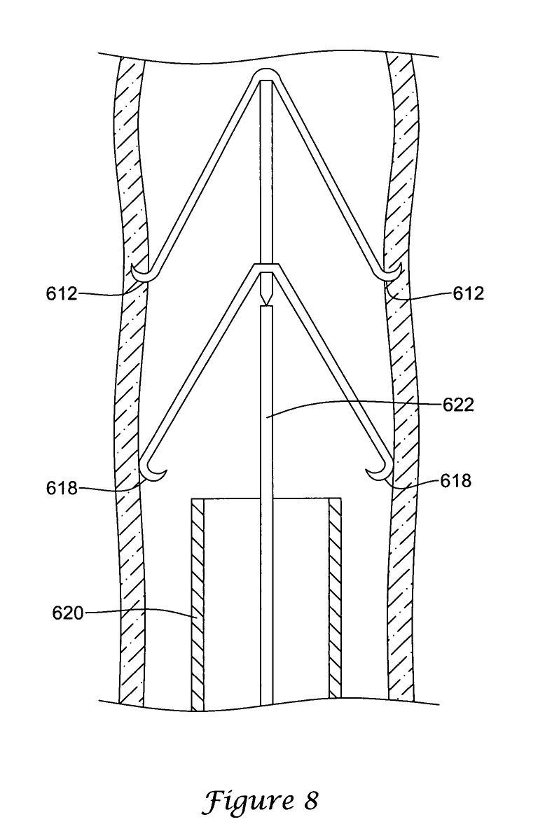

FIG. 7 is a diagrammatic view of an intracorporal filter assembly 600 loaded in a delivery device. Assembly 600 includes a first filter 602, a second filter 604, and an elongate member 606 extending from the first filter, and on which second filter 604 is releasable disposed. Filter 602 includes apical head 608, legs 610, and anchoring members 612, and filter 604 includes apical ring 614, legs 616, and atraumatic ends 618. Legs 610 and 618 are compressed into a delivery configuration by delivery catheter 620. The delivery system also may include a pushing mechanism 622, which may act either against the elongate member as shown or against atraumatic ends 618. As shown in FIG. 8, the assembly is deployed from the delivery device. This may occur through the advancement of pushing mechanism 622 or through the withdrawal of delivery catheter 620. When assembly 600 leaves the delivery catheter, legs 610 and 618 expand to their deployed configurations. Anchoring members 612 engage the vessel wall and atraumatic ends rest against the vessel wall. Once the delivery device has been retracted, assembly 600 is deployed and filters emboli.

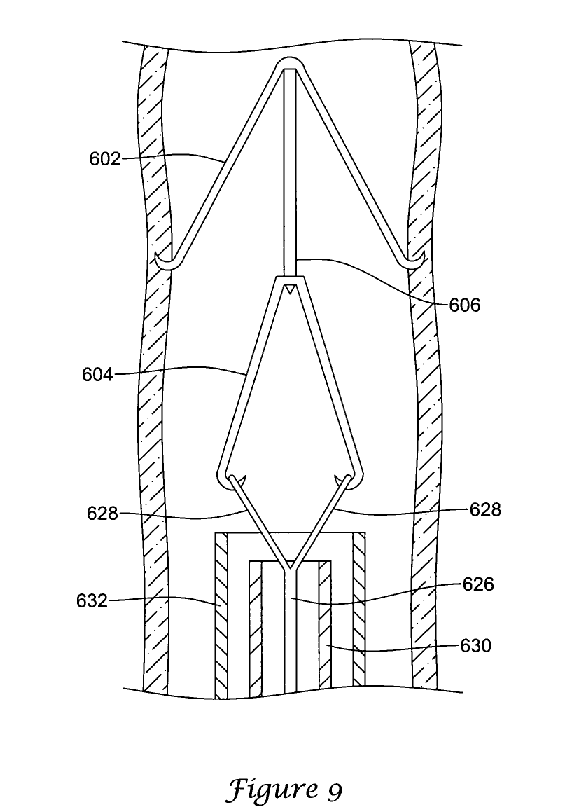

If it is desired to remove filter 604, for example the filter is filled with emboli, the filter may be withdrawn as shown in FIG. 9, which is a diagrammatic view of a filter 604 being withdrawn from assembly 600. A retrieval device 626 has hooks on the ends of arms 628 to engage the hooks on atraumatic ends 618. A catheter 630 is sized to compress the arms of retrieval device 626 as it is advanced. Thus, filter 604 may be compressed for retrieval. Once filter 604 is ready for retrieval, it may be pulled off elongate member 606 and into catheter 632 and out from the body. This is one exemplary retrieval mechanism. Other retrieval mechanisms may be used and the filters may include other structures to engage the retrieval mechanisms. For example, filter 604 may include a loop slidably attached to arms 616 that pulls the arms together when pulled.

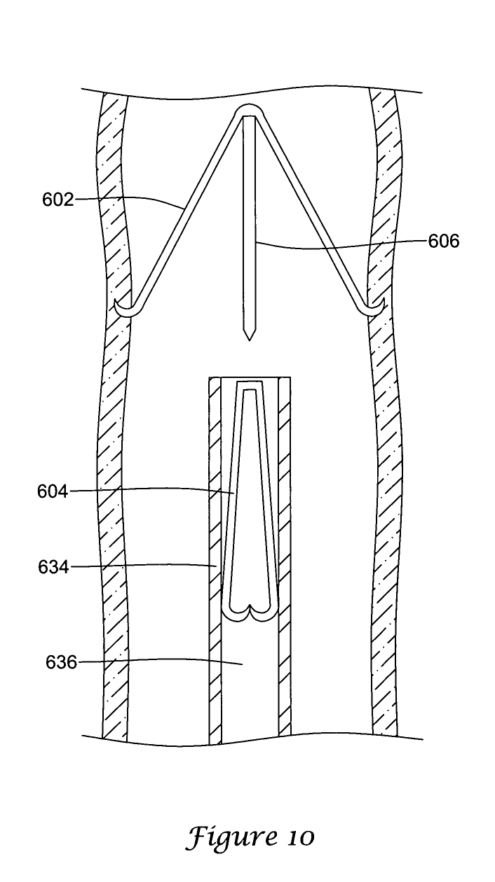

It may be desirable to replace filter 604 with a new filter. FIG. 10 is a diagrammatic view of a filter 604 loaded in a delivery device that has been advanced to a position proximate elongate member 606. The delivery device includes a catheter 634 and a pusher 636. Once filter 604 is in position, pusher 636 advances filter 604 on elongate member 606. Catheter 634 may be withdrawn to allow filter 604 to spring into place. Pusher 636 may then be withdrawn as well.

Numerous advantages of the invention covered by this document have been set forth in the foregoing description. It will be understood, however, that this disclosure is, in many respects, only illustrative. Changes may be made in details, particularly in matters of shape, size, and arrangement of parts without exceeding the scope of the invention. The invention's scope is, of course, defined in the language in which the appended claims are expressed.

What is claimed is:

1. An intravascular filter assembly for use in a body vessel lumen having a wall, comprising:
a first filter having a mouth facing in a first direction at a first location for receiving emboli and an end in a second direction opposite the first direction; and
a second filter having a mouth facing in the first direction at a first location for receiving emboli and an end in the second direction;
wherein the second filter is removably attached to the first filter.

2. The intravascular filter assembly of claim 1, wherein the first filter has anchoring members for retaining the first filter with respect to a wall of a body vessel lumen.

3. The intravascular filter assembly of claim 1, wherein the second filter end is in the first direction from the first filter mouth.

4. The intravascular filter assembly of claim 1, wherein the second filter end is in the second direction from the first filter mouth.

5. The intravascular filter assembly of claim 1, wherein the first filter comprises a cavity and wherein the second filter end is disposed in the first filter cavity.

6. The intravascular filter assembly of claim 1, wherein the first filter has a first set of legs extending radially outward at a first set of radial angles, and wherein the second filter has a second set of legs extending radially outward at the first set of radial angles.

7. The intravascular filter assembly of claim 1, wherein the first filter has a first set of legs extending radially outward at a first set of radial angles, and wherein the second filter has a second set of legs extending radially outward at a second set of radial angles different from the first set of radial angles.

8. The intravascular filter assembly of claim 1, wherein the first filter has a first set of legs and the second filter has a second set of legs radially offset from the first set of legs.

9. The intravascular filter assembly of claim 1, wherein the first filter has a first radial extent when unconstrained, and wherein the second filter has a second radial extent when unconstrained, the second radial extent being less than the first radial extent.

10. The intravascular filter assembly of claim 9, wherein the second filter is configured to have a radial extent less than that of the wall of the body vessel lumen.

11. The intravascular filter assembly of claim 1, wherein the second filter has ends configured to rest on the wall of the body vessel lumen.

12. The intravascular filter assembly of claim 11, wherein the second filter has ends that curve inwards.

13. The intravascular filter assembly of claim 11, wherein the second filter has ends configured to lie flat against the wall of the body vessel lumen.

14. The intravascular filter assembly of claim 11, wherein the second filter has hooks configured to release from the wall of the body vessel lumen when the second filter is pulled in the first direction.

15. The intravascular filter assembly of claim 1, further comprising a third filter disposed in the first direction from the second filter and releasably attached thereto.

16. The intravascular filter assembly of claim 1, wherein the first filter comprises a central longitudinal member and wherein the second filter is releasably attached to the central longitudinal member.

17. The intravascular filter assembly of claim 16, wherein the second filter comprises a ring proximate the end for disposal on the central longitudinal member.

18. The intravascular filter assembly of claim 16, wherein the central longitudinal member has a decreasing taper in the first direction.

19. The intravascular filter assembly of claim 16, wherein the central longitudinal member has step-wise cross-sectional reductions in the first direction.

20. The intravascular filter assembly of claim 1, wherein the second filter comprises a central longitudinal member and wherein the central longitudinal member is releasably attached to the first filter.

21. The intravascular filter assembly of claim 1, wherein the first filter comprises a first central longitudinal elongate member and wherein the second filter comprises a second central longitudinal member releasably attached to the first central longitudinal elongate member.

22. The intravascular filter assembly of claim 1, wherein the second filter comprises an element to interface with a retractor.

23. The intravascular filter assembly of claim 22, wherein the element is aknob.

24. The intravascular filter assembly of claim 22, wherein the element is a loop.

25. The intravascular filter assembly of claim 22, wherein the element is a hook.

26. The intravascular filter assembly of claim 22, wherein the element is a screw thread.

27. The intravascular filter assembly of claim 1, wherein the second filter comprises a mechanism for moving the filter to a compressed state.

28. The intravascular filter assembly of claim 27, wherein the mechanism is one or more actuators.

29. The intravascular filter assembly of claim 27, wherein the mechanism is a loop attached to the second filter proximate the mouth.

30. The intravascular filter assembly of claim 29, wherein the loop is configured to be narrowed by pulling on a portion thereof.

31. The intravascular filter assembly of claim 1, wherein the second filter at least partially nests inside the first filter cavity, wherein the first filter comprises three legs extending from the first filter end to the first filter mouth, wherein the second filter comprises three legs extending from the second filter end to the second filter mouth.

32. The intravascular filter assembly of claim 31, wherein the second filter legs are radially offset from the first filter legs.

33. The intravascular filter assembly of claim 31, wherein the first filter comprises no more than three legs and the second filter comprises no more than three legs.

34. The intravascular filter assembly of claim 31, wherein the first filter comprises no more than four legs and the second filter comprises no more than four legs.

35. The intravascular filter assembly of claim 1, wherein the second filter is in the first direction from the first filter mouth, wherein the first filter comprises five legs extending from the first filter end to the first filter mouth, wherein the second filter comprises five legs extending from the second filter end to the second filter mouth.

36. The intravascular filter assembly of claim 35, wherein the first filter comprises six legs and wherein the second filter comprises six legs.

37. An intravascular filter kit, comprising:
a first filter having a mouth, a cavity for receiving emboli and anchoring members for anchoring the first filter to the wall of a body vessel lumen; and
a second filter having a mouth and a cavity for receiving emboli,
wherein the first filter and the second filter are configured to be releasably attached to each other.

38. The kit of claim 31, further comprising a third filter having a mouth and a cavity, the third filter and the first filter configured to be releasably attached to each other.

39. The kit of claim 31, wherein the second filter is configured to nest at least partially inside the cavity of the first filter.

40. A method of filtering emboli in a blood vessel having a wall, comprising the steps of:
providing a first filter having a mouth, a cavity and anchoring members;
anchoring the first filter to the wall;
providing a second filter having a mouth and a cavity; and
disposing the second filter in the body vessel lumen near the first filter.

41. The method of claim 40 wherein the steps of anchoring the first filter and disposing the second filter occur simultaneously.

42. The method of claim 40 further comprising the step of removing the second filter from the body vessel lumen while keeping the first filter in place.

43. The method of claim 40 further comprising the step of treating a region of interest in the body vessel lumen, and wherein the step of anchoring the first filter includes the step of anchoring the first filter on a first side of the region of interest; and wherein the step of disposing the second filter includes the step of disposing the second filter on the first side of the region of interest.

44. The method of claim 40, further including the step of releasably attaching the second filter to the first filter.

45. An intravascular filter assembly, comprising:
a first filter having a mouth, a cavity and anchoring members; and
a second filter disposed in the cavity.
